(19) United States
(12) Patent Application Publication
(10) Pub. No.: US 2018/0017551 A1
(43) Pub. Date: Jan. 18, 2018

(54) PROCESS FOR MAKING AN ARRAY

(71) Applicants: AGENCY FOR SCIENCE, TECHNOLOGY AND RESEARCH, Singapore (SG); National University of Singapore, Singapore (SG)

(72) Inventors: Bee Khuan Jaslyn LAW, Singapore (SG); Hong Yee LOW, Singapore (SG); Mona SURYANA, Singapore (SG); Kee Fong WEE, Singapore (SG); Michael Patrick SHEETZ, Singapore (SG); King Fai Evelyn YIM, Singapore (SG); Zheng Jie NG, Singapore (SG); Kim Kiat TEO, Singapore (SG); Lance Cameron KAM, New York, NY (US)

(73) Assignees: AGENCY FOR SCIENCE, TECHNOLOGY AND RESEARCH, Singapore (SG); National University of Singapore, Singapore (SG)

(21) Appl. No.: 15/611,498

(22) Filed: Jun. 1, 2017

Related U.S. Application Data
(62) Division of application No. 13/635,631, filed on Oct. 2, 2012, now abandoned.
(60) Provisional application No. 61/314,871, filed on Mar. 17, 2010.

(51) Int. Cl. G01N 33/543; B82Y 30/00; B01J 19/00

(52) U.S. Cl. CPC G01N 33/54366; B01J 19/0046; B82Y 30/00; B01J 2219/0061; B01J 2219/00621; B01J 2219/00743; B01J 2219/00659

(57) ABSTRACT

There is disclosed a method of making an array for cell assays comprising the step of providing an array of structures on a substrate, each of said structures having a pre-defined topography thereon, and wherein at least one structure has a different topography from at least one other structure.

### CROSS-REFERENCE TO RELATED APPLICATIONS

The present application is a divisional of U.S. application Ser. No. 13/635,631 filed on Oct. 2, 2012, entitled “A Process for Making an Array,” which is a 35 U.S.C. §371 national stage application of PCT/SG2011/000106 filed on Mar. 17, 2011, entitled “A Process for Making an Array,” which claims benefit of U.S. provisional application No. 61/314,871 filed on Mar. 17, 2010, all of which are incorporated herein by reference in their entirety.

### TECHNICAL FIELD

The present invention generally relates to a method of making an array. The present invention also relates to an array and uses thereof.

### BACKGROUND

Understanding how surface topography affects cellular functions is integral in developing purpose-specific cell regulating cues in various biomedical applications. For example, the interaction of T-cells with antigen presenting cells (APCs) is critical for the coordination of the immune system and the activation of T-cells is often studied using planar surfaces in place of APCs.

Presently, the main obstacle facing cell-topographical studies is the lack of a single surface possessing a large range of different topographies, such as different heights, dimensions and feature shapes, in order to facilitate fast microscopy-based screening and subsequent analysis of the data generated.

The lack of such multi-architectural surfaces is due to the fact that there is no one single patterning technique that can be used to simultaneously fabricate multiple surface topographical features onto a single substrate. All existing lithography techniques are limited to specific geometries and resolutions. Therefore, different techniques have to be used for each specific geometry, resolution and aspect ratio. For example: optical lithography for micro-feature resolutions; electron-beam lithography for nano-feature resolutions; gray mask lithography for different aspect ratio patterns; optical lithography in combination with isotropic etching for curved features; and interference lithography for three-dimensional structures. Thus, in order to create a multi-architectural topographical surface on a single substrate, each technique needs to be employed separately and it is also necessary to mask all the other patterns whilst each pattern is being fabricated. This task is clearly challenging and not practical if a large number of vastly different topographies are involved.

Accordingly, there is a need to provide method for making an array comprising multiple topographies on a single substrate that overcomes, or at least ameliorates, the disadvantages mentioned above.

### SUMMARY

According to a first aspect, there is provided a method of making an array for cell assays comprising the step of providing an array of structures on a substrate, each of said structures having a defined topography thereon, and wherein at least one structure has a different topography from at least one other structure.

It is an advantage of the method described herein that a plurality of different topographies can be presented on a single substrate to form a microarray, which would otherwise be difficult using existing single patterning techniques.

It is a further advantage of the method described herein that different topographies can be presented on a single substrate which permits customization of the array.

Advantageously, the method described herein provides a low cost fabrication process. In particular, nanoimprint technology, which is a cost-efficient, scalable, high throughput nanoimprinting technique, may be used to create the different topographies which are otherwise prohibitively expensive if they are fabricated by conventional electron beam lithography.

It is a further advantage that the method as described herein may provide a biocompatible array.

It is a further advantage of the method as described herein that the array may be replicated using known casting methods, for example PDMS casting. This provides a cost-effective way of producing replicas of the original array.

Advantageously, the method as described herein may not encounter problems of resolution limit in providing the different topographies, as nanoimprinting may be used to form the topographies, where the limit of resolution is limited by the mold to be used for imprinting.

It is a further advantage of the method as described herein that the individual topographies of the array can be optimized as different topographies are imprinted separately.

It is a further advantage of the method as described herein that a dry/etch-free method may be used to form the different topographies on a single substrate.

Advantageously, the method as described herein permits the use of different types of polymer (for example, PMMA, PC, ETFE and the like) to form the structures having a defined topography thereon.

According to a second aspect there is provided an array comprising a substrate having an array of structures projecting therefrom, wherein each of said structures have a defined topography thereon, and wherein at least one structure has a different topography from at least one other structure. In one embodiment, the array can be used as a master mold to make other array structures, such as by imprint lithography.

### Definitions

While most of the terms used herein will be recognizable to those of skill in the art, the following definitions are nevertheless put forth to aid in the understanding of the present invention. It should be understood, however, that when not explicitly defined, terms should be interpreted as adopting a meaning presently accepted by those of skill in the art.

The term “micro-structure,” as used herein relates to structures that have dimensions that are in the “microscale” size range.

The term “nano-structure,” as used herein relates to structures that have dimensions that are in the “nanoscale” size range.

The term “structure density” in the context of this specification refers to the number of structures patterned or disposed within a defined area on a substrate.

The term “array” generally refers to multiple numbers of structures distributed within an area and spaced apart, unless otherwise indicated. Structures within an array all do not have to have the same orientation.

The term “ordered array” generally refers to the placement of elements in a specified or predetermined pattern where the elements have distinct spatial relationships to one another. Hence, the term “ordered array” generally refers to structures distributed within an area with distinct, specified or predetermined spatial relationships to one another. For example, the spatial relationships within an ordered array may be such that the structures are spaced apart from one another by generally equal distances. Other ordered arrays may use varying, but specified or predetermined, spacings.

The term “three-dimensional,” abbreviated “3-D” and as defined herein, refers to structures or structural features that vary (structurally) with depth.

The term “spin-coating,” as defined herein, generally refers to a process wherein a polymer solution is dispersed on a surface (e.g., a mold) and the surface is rapidly spun centrifugally to thereby force the solution to spread out on a surface and form a thin layer of de-solvated polymer in the process.

The term “glass transition temperature” (T_{g}) is to be interpreted to include any temperature of a polymer at which the polymer lies between the rubbery and glass states. This means that above the T_{g}, the polymer becomes rubbery and can undergo elastic or plastic deformation without fracture. Above this temperature, such polymers can be induced to flow under pressure. When the temperature of the polymer falls below the T_{g}, generally, the polymer will become more inflexible but can be deformed when a stress is applied to the polymer. It should be noted that the T_{g} is not a sharp transition temperature but a gradual transition and is subject to some variation depending on the experimental conditions (e.g., film thickness, tacticity of the polymer, etc.). The actual T_{g} of a polymer film will vary as a function of film thickness. The T_{g} will be defined herein as being the bulk glass-transition temperature of the polymer substrate. The bulk glass transition temperature is a specific value that is widely agreed upon in the literature. Glass transition temperature values of polymers may be obtained from PPP Handbook™ software edited by Dr D. T Wu, 2000.

The term “imprint” and grammatical variations thereof, in the context of this specification, is intended to cover any form of physical impression that has been made in a pliable solid body, such as a thermoplastic polymer substrate. An imprint may form a structure on a substrate and typically, an imprint is a generally elongate structure that extends from the surface of a substrate along a longitudinal axis extending between a proximal end disposed on or adjacent to the substrate and a distal end opposite to the proximal end. Typically, the longitudinal axis is generally normal relative to a planar axis of the substrate but the longitudinal axis may be varied significantly such as at an angle of 45° from a planar axis of the substrate.

In an array of imprints that have been orderly formed as a series of rows and columns on a substrate, trenches may be formed between the adjacent rows. The imprint may be in the nanoscale or microscale size range both in their length dimension and thickness dimension, and hence the trenches may also be in the nanoscale or microscale size range.

The term “nanoimprinting lithography” is to be interpreted broadly to include any method for printing or creating a pattern or structure on the microscale and/or nanoscale size range on the surface of a substrate by contacting a mold with the defined pattern or structure on the surface at certain temperatures and pressures.

The term “thermoplastic” refers to a material that softens when sufficient heat and pressure is applied to the material and hardens as it cools down. When the term “thermoplastic” is used to refer to a polymer, the thermoplastic polymer softens at a temperature above the T₉ and hardens when the temperature is decreased until below the T_{g}. As the thermoplastic polymer cools from the T_{g}, the thermoplastic polymer becomes less moldable or deformable as compared to the state of the thermoplastic polymer in the first applying step (a) when the mold is applied to the polymer substrate.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a method of making an array for cell assays will now be disclosed. The method comprises the step of step of providing an array of structures on a substrate, each of said structures having a defined topography thereon, and wherein at least one structure has a different topography from at least one other structure.

In one embodiment, the array of structures may extend from a planar surface of the substrate. In one embodiment, the structures may be generally elongate and have a proximal end that extends from the substrate and a distal end that is opposite to said proximal end. In another embodiment, the topography of said structures is provided on said proximal end.

In one embodiment the elongate structures have a longitudinal axis extending from their proximal end to their distal end and wherein the longitudinal axis is generally normal to a planar surface of said substrate. In one embodiment, the structures are in the form of imprints. In another embodiment, the structures are generally rectangular, square, triangular, or cylindrical in shape when viewed in cross section in a plane normal to said longitudinal axis. In one embodiment, the structures are generally cylindrical in shape.

In another embodiment, the providing step comprises the step of adhering said structures to said substrate. The adhering step may comprise the step of providing an adhesive layer on said substrate before said adhering step. In one embodiment, the adhesive layer may be selected from PDMS or optical adhesives that permit bonding of thermoplastic materials onto solid substrates such as silicon or glass.

In one embodiment, the adhesive layer is a polymeric solution. The polymer solution may be a silane-containing compound. The silane-containing compound may be selected from the group consisting of 2-mercaptoethyl trimethoxysilane, 3-mercaptopropyl trimethoxysilane, 2-mercaptopropyl triethoxysilane, 3-mercaptopropyl triethoxysilane, 2-mercaptoethyl tripropoxysilane, 2-mercaptoethyl tri sec-butoxysilane, 3-mercaptopropyl tri-t-butoxysilane, 3-mercaptopropyl triisopropoxysilane, 3-mercaptopropyl trioctoxysilane, 2-mercaptoethyl tri-2′-ethylhexoxysilane, 2-mercaptoethyl dimethoxyethoxysilane, 3-mercaptopropyl methoxyethoxypropoxysilane, 3-mercaptopropyl dimethoxy methylsilane, 3-mercaptopropyl methoxy dimethylsilane, 3-mercaptopropyl ethoxy dimethylsilane, 3-mercaptopropyl diethoxy methylsilane, 3-mercaptopropyl cyclohexoxydimethylsilane, 4-mercaptobutyl trimethoxysilane, 3-mercapto-3-methylpropyltrimethoxysilane, 3-mercapto-3-methylpropyl-tripropoxysilane, S-mercapto-S-ethylpropyl-dimethoxy methylsilane, 3-mercapto-2-methylpropyl trimethoxysilane, 3-mercapto-2-methylpropyl dimethoxy phenyl silane, S-mercaptocyclohexyl-triinethoxysilane, 12-mercaptododecyl trimethoxy silane, 12-mercaptododecyl triethoxy silane, 18-mercaptooctadecyl trimethoxysilane, 18-mercaptooctadecyl methoxy dim ethyl silane, 2-mercapto-2-methylethyl-tripropoxysilane, 2-mercapto-2-methylethyl-trioctoxysilane, 2-mercaptophenyl trimethoxysilane, 2-mercaptophenyl triethoxysilane, 2-mercaptotolyl trimethoxysilane, 2-mercaptotolyl triethoxysilane, 1-mercaptomethyltolyltrimethoxysilane, 1-mercaptomethyltolyl triethoxysilane, 2-mercaptoethylphenyl trimethoxysilane, 2-mercaptoethylphenyl triethoxysilane, 2-mercaptoethyltolyl trimethoxysilane, 2-mercaptoethyltolyl triethoxysilane, 3-mercaptopropylphenyl trimethoxysilaneand, 3-mercaptopropylphenyl triethoxysilane, and the aminosilanes 3-aminopropyltrimethoxysilane, 3-aminopropyltriethoxysilane, 4-aminobutyltriethoxy-silane, N-methyl-3-amino-2-methylpropyltrimethoxysilane, N-ethyl-3-amino-2-methylpropyltrimethoxysilane, N-ethyl-3-amino-2-methylpropyldiethoxymethylsilane, N-ethyl-3-amino-2-methylpropyltriethoxysilane, N-ethyl-3-amino-2-methylpropyl-methyldimethoxysilane, N-butyl-3-amino-2-methylpropyltrimethoxysilane, 3-(N-methyl-2-amino-1-methyl-1-ethoxy)-propyltrimethoxysilane, N-ethyl-4-amino-3, 3-dimethylbutyldimethoxymethylsilane, N-ethyl-4-amino-3, 3-dimethylbutyltrimethoxy-silane, N-(cyclohexyl)-3-aminopropyltrimethoxysilane, N-(2-aminoethyl)-3-aminopropyltrimethoxysilane, N-(2-aminoethyl)-3-aminopropyltriethoxy-silane, N-(2-aminoethyl)-3-aminopropylmethyldimethoxysilane, aminopropyltriethoxysilane, bis-(3-trimethoxysilyl-2-methylpropyl) amine, N-(3′-trimethoxysilylpropyl)-3-amino-2-methylpropyltrimethoxysilane and polydimethylsiloxane.

In one embodiment the silane-containing compound comprises polydimethylsiloxane (PDMS). In one embodiment, the polydimethylsiloxane solution is Sylgard®184. In an exemplary embodiment, the Sylgard@184 comprises an elastomer and curing agent. The PDMS solution may be formed by mixing the elastomer and the curing agent in a ratio from 10:1 to 3:1 (elastomer:curing agent). In one embodiment the ratio is 3:1. Advantageously, different thicknesses of the adhesive layer during spin-coating onto the substrate can be obtained using different ratios of the elastomer and curing agent.

In one embodiment, the providing step comprises spin-coating the polymeric solution onto the substrate. The step of spin coating permits the PDMS polymer solution to be coated planarly onto the said substrate. The structures are then adhered on the said substrate via the PDMS polymer. In one embodiment, the step of spin coating comprises removing the polymer solution while said array of structures are disposed in said polymer solution to form a polymer layer that adheres said structures to said substrate. In one embodiment, the step of removing the solvent from the removing step comprises heating the polymeric solution.

In another embodiment, the array of structures adhered onto the said substrate via the PDMS polymer are heated to cure and harden the PDMS polymer to firmly attach the said array of structures onto the said substrate. In one embodiment heating step is performed at a temperature range of from 30° C. to 100° C. In another embodiment, the heating step is performed at a temperature of about 30° C., about 35° C. about 40° C. about 45° C. about 50° C., about 55° C., about 60° C., about 65° C., about 70° C., about 75° C., about 80° C., about 85° C., about 90° C., about 95° C. or about 100° C. In one embodiment, the heating step is carried out at 70° C. The heating step may be carried out for a time selected from a range of from 30 mins to 24 hours. In one embodiment, the heating step is carried out for a time selected from about 30 mins, about 45 mins, about 1 hour, about 1 hour 30 mins, about 2 hours, about 4 hours, about 6 hours, about 8 hours, about 10 hours, about 12 hours, about 14 hours, about 16 hours, about 18 hours, about 20 hours, about 22 hours or about 24 hours. In one embodiment, the heating step is carried out for 1 hour.

In one embodiment, the heating step is performed under vacuum conditions. Advantageously, this removes any residual bubbles under the structures when they are applied to the polymeric solution.

In another embodiment, the topography is formed by formations in the microscale or the nanoscale size range.

In another embodiment, the method as described herein comprises, before said providing step, the step of forming said topographic formations on a sacrificial substrate. In one embodiment, the sacrificial substrate is a polymer film. The polymer may be a thermoplastic polymer. The first and/or second substrates may be a polymer substrate such as a thermoplastic polymer substrate.

The sacrificial thermoplastic polymer substrate may comprise at least one monomer selected from the group consisting of acrylates, phthalamides, acrylonitriles, cellulosics, styrenes, alkyls, alkyl methacrylates, alkenes, halogenated alkenes, amides, imides, aryletherketones, butadienes, ketones, esters, acetals, carbonates and combinations thereof In one embodiment, the thermoplastic polymer is selected from poly carbonate and poly methyl methacrylate.

During the forming step, the resultant formations on the sacrificial substrate are provided by the step of contacting the sacrificial substrate with a mold.

During the contacting step, the resultant topography on the sacrificial substrate is of complementary configuration to that of the mold. For example, if the pattern on the mold comprises gratings, the gratings may result in channels of complementary configuration on the sacrificial substrate when the mold is applied to the sacrificial substrate.

In one embodiment, during the step of contacting the mold to the sacrificial substrate, the method may comprise the step of selecting a temperature that is above the glass transition temperature (Tg) of the sacrificial substrate. At this temperature, the polymer softens and may conform to the shape of the mold such that an imprint is created on the surface of the polymer whereby the pattern of the imprint may be of complementary configuration to the pattern on the mold when the polymer is cooled and subsequently hardens. Furthermore, the mold may be preferably applied at a predetermined pressure for a certain period of time to form an imprint on the surface of the sacrificial substrate. The temperature and pressure to be applied will be dependent on the polymer used and the topography of the mold.

The molds may be made of any suitable material that is chemically inert and may be harder than the softened substrate when used at the temperature that is above the glass transition temperature of the substrate. The molds may be made of silicon, a metal, a glass, quartz, a ceramic or a combination thereof.

The temperature used when applying a substrate to a mold is dependent on the type of thermoplastic polymer used. Typically, the temperature may be selected from the group consisting of about 120° C. to about 200° C., about 140° C. to about 200° C., about 160° C. to about 200° C., about 180° C. to about 200° C., about 120° C. to about 140° C., about 120° C. to about 160° C., about 120° C. to about 180° C. and about 150° C. to about 160° C. The temperature selected is preferably a temperature greater than that of the glass transition temperature of the sacrificial substrate. Preferably, the temperature selected is approximately at least 20° C. greater than the glass transition temperature of the sacrificial substrate. The pressure used when contacting the mold to a surface of the sacrificial substrate may be independently selected from the group consisting of about 10 bar (1 MPa) to about 60 bar (6 MPa), 10 bar (1 MPa) to about 50 bar (5 MPa), about 10 bar (1 MPa) to about 40 bar (4 MPa), about 10 bar (1 MPa) to about 30 bar (3 MPa), about 10 bar (1 MPa) to about 20 bar (2 MPa).

The time period used when applying the sacrificial substrate to the mold is dependent on the type of thermoplastic polymer used and the complexity of the imprint forming surface. Typically, the time period may be in the range of about 5 minutes to about 45 minutes, about 5 minutes to about 15 minutes, about 5 minutes to about 30 minutes, about 15 minutes to about 45 minutes, about 30 minutes to about 45 minutes and about 10 minutes to about 30 minutes.

It is an advantage of the method as described herein that as different topographies are imprinted separately, imprinting conditions can be optimized with respect to specific designs. For example, in one embodiment, where imprints for topographies such as gratings, dimples, and curved lens structures a single imprinting condition is used comprising a temperature of 180° C. at 60 Bar for 600 seconds. For more delicate structures such as pillars a preheating step may be performed, prior to imprinting, at 180° C. for 120 seconds before imprinting at 180° C. at 40 Bar for 300 seconds. For complex hierarchical structures a sequential imprinting process may be employed. For example, a first imprinting process at 180° C. at 60 Bar for 600 seconds and a second imprinting process at 140° C. at 40 Bar for 300 seconds.

In one embodiment the step of forming said topographic formations comprises the step of providing the sacrificial substrate with an array of the topographic structures in the microscale or nanoscale size range. In another embodiment, the array of microscale or nanoscale topographic structures on said sacrificial substrate are selected from the group consisting of trenches, pillars, gratings, dimples, wells, curved lens structures, other 3-D structures and hierarchical 3-D structures.

The height of the topographic formations may be in the microscale. The height of the topographic formations may be selected from the group consisting of about 1 micron to about 10 microns, about 1 micron to about 3 microns; about 1 micron to about 5 microns, about 1 micron to about 7 microns, about 1 micron to about 9 microns, about 3 microns to about 10 microns, about 5 microns to about 10 microns, about 7 microns to about 10 microns and about 9 microns to about 10 microns.

In another embodiment, the height of the topographic formations may be in the nanoscale. The height of the topographic formations may be selected from the group consisting of about 10 nm to about 1000 nm, about 10 nm to about 500 nm, about 10 nm to about 300 nm; about 10 nm micron to about 250 nm, about 10 nm to about 200 nm, about 10 nm to about 150 nm, about 10 nm to about 100 nm.

In one embodiment, the topographical formations may have an associated pitch. The pitch may be in the microscale and be selected from the group consisting of from about 0.5 microns to about 15 microns, about 1 micron to about 12 microns, about 2 microns to about 10 microns and about 5 microns to about 10 microns. Alternatively, the pitch may be in the nanoscale and be selected from the group consisting of from about 100 nm to about 1000 nm, about 100 nm to about 500 nm, about 100 nm to about 250 nm, about 100 nm to about 200 nm and about 100 nm to about 150 nm.

In another embodiment, the width of the topographic formations may be in the nanoscale. The width of the topographic formations may be from 50 nm to about 1000 nm. In another embodiment, the width of the topographic formations may be selected from the group consisting of about 50 nm to about 1000 nm, about 50 nm to about 800 nm, about 50 nm to about 600 nm, about 50 nm to about 400 nm, about 50 to about 300, about 100 nm to about 260 nm, about 100 nm to about 140 nm, about 100 nm to about 180 nm, about 100 nm to about 220 nm, about 100 nm to about 260 nm, about 260 nm to about 300 nm, about 220 nm to about 300 nm, about 180 nm to about 300 nm, about 140 nm to about 300 nm and about 150 nm to about 250 nm. Where the topographic formation is cylindrical in shape, the width of the topographic formation refers to the diameter of the cylindrical-shaped topographic formation.

In another embodiment, the width of the topographic formations may be in the microscale. The width of the topographic formations may be selected from the group consisting of about 1 micron to about 30 microns, about 1 micron to about 20 microns, about 1 micron to about 15 microns, about 1 micron to about 10 microns, about 1 micron to about 5 microns; about 1 micron to about 4 microns, about 1 micron to about 3 microns, about 1 micron to about 2 microns.

The width of the gratings and/or trenches may be independently selected from the group consisting of about 1 microns to about 50 microns, about 2 microns to about 40 microns, about 5 microns to about 30 microns, about 5 microns to about 20 microns, about 5 microns to about 10 microns, about 10 microns to about 50 microns, about 20 microns to about 50 microns, about 30 microns to about 50 microns and about 40 microns to about 50 microns.

In another embodiment, the width of the gratings and/or trenches may be independently selected from the group consisting of about 100 nm to about 1000 nm, about 200 nm to about 500 nm and about 250 nm to about 500 nm.

The grating formation may have a grating constant in the range of 100 nm to 1000 nm.

In one embodiment, the height of the grating formation may be in the range of about 10 to about 1000 nm. The grating formation may have a shape, when viewed in cross-section, selected from the group consisting of sinusoidal wave shape, square wave, trapezoidal shape, blazed shape and triangular shape.

The gratings and/or trenches may have an aspect ratio selected from the group consisting of about 0.1 to about 3.0, about 0.1 to about 2.5, about 0.1 to about 2.0, about 0.1 to about 1.5, about 0.1 to about 1.0, and about 0.1 to about 0.5. In one embodiment, the aspect ratio of the gratings and/or trenches may be about 0.5.

In a another embodiment the topographic formation may comprise a line and space arrangement wherein the width of the line is selected from the group consisting of about 1 μm, about 2 μm, about 3 μm, about 4 μm and about 5 μm. Alternatively, the width of the line is selected from the group consisting of about 100 nm, about 150 nm, about 200 nm, about 250 nm about 300 about 350 nm about 400 nm and about 500 nm. The width of the space is selected from the group consisting of about 1 μm, about 2 μm, about 3 μm, about 4 μm and about 5 μm. Alternatively, the width of the space is independently selected from the group consisting of about 100 nm, about 150 nm, about 200 nm, about 250 nm, about 300 about 350 nm, about 400 nm and about 500 nm.

In another embodiment, the topographic formations may comprise pillars and/or circular holes and/or wells. The diameter of the pillars and/or circular holes and/or wells may be in the microscale. The topographic formations may also comprise pillars and/or dimples and/or lens structures. The diameter of the columns and/or circular holes and/or dimples and/or lens structures may be independently selected from the group consisting of about 1 micron to about 10 microns, about 1 micron to about 8 microns, about 1 micron to about 6 microns, about 1 micron to about 4 microns, about 1 micron to about 2 microns, about 2 microns to about 10 microns, about 4 microns to about 10 microns, about 6 microns to about 10 microns, about 8 microns to about 10 microns and about 4 microns to about 6 microns. In one embodiment, the diameter of the columns and/or circular holes may be about 5 microns.

The diameter of the pillars and/or circular holes and/or wells may be in the nanoscale. The topographic formations may also comprise pillars and/or dimples and/or lens structures. The diameter of the columns and/or circular holes and/or dimples and/or lens structures may be independently selected from the group consisting of about 100 nm to about 1000 nm, about 200 nm to about 500 nm and about 250 nm to about 500 nm.

The columns, pillars, dimples and/or circular holes and/or lens structures may have an aspect ratio selected from the group consisting of about 0.1 to about 2.0, about 0.1 to about 1.5, about 0.1 to about 1.0, and about 0.1 to about 0.5.

In one embodiment the topographical formations may have an aspect ratio selected from the group consisting of about 5 to about 10, about 5 to about 7, about 5 to about 9, about 7 to about 10, about 9 to about 10 and about 5 to about 7. In one embodiment the topographical formations may have an aspect ratio selected from the group consisting of about 0.1 to about 5, about 0.1 to about 4, about 0.1 to about 3, about 0.1 to about 2, about 0.1 to about 1, about 1 to about 3, about 1 to about 2, about 0.1 to about 1.5, about 0.1 to about 1.0, and about 0.1 to about 0.5.

In one embodiment, the method further comprises the step of removing a portion of said microscale or nanoscale topographic structures while attached to said sacrificial substrate. In one embodiment, the step of removing a portion of said microscale or nanoscale topographic structures while attached to said sacrificial substrate comprises cutting or punching.

In one embodiment, step of removing a portion of said microscale or nanoscale topographic structures while attached to said sacrificial substrate comprises driving a punch through said sacrificial substrate.

In another embodiment, the providing step comprises providing an ordered array of structures on said substrate. In one embodiment, said ordered array is in the form of a matrix, preferably consisting of 1 to “n” number of rows and 1 to “m” number of columns, wherein n and m are integers. In one embodiment, there n is a value from 2 to 10, from 2 to 10, from 2 to 20, from 2 to 30, from 2 to 40, from 2 to 50, from 2 to 60, from 2 to 70, from 2 to 80, from 2 to 90 or from 2 to 100. In another embodiment, there m from 2 to 10, from 2 to 20, from 2 to 30, from 2 to 40, from 2 to 50, from 2 to 60, from 2 to 70, from 2 to 80, from 2 to 90 or from 2 to 100.

In one embodiment, the array has a structure density of 2 to 10, from 2 to 20, from 2 to 30, from 2 to 40, from 2 to 50, from 2 to 60, from 2 to 70, from 2 to 80, from 2 to 90 or from 2 to 100 structures in a defined area of the array.

In one embodiment, the diameter of each structure is in the range of from 100 μm to 5 mm, from 500 μm to 5 mm, from 1 mm to 5 mm or from 1 mm to 2 mm. Advantageously, the the size of the structures can be customized to meet particular requirements. In one embodiment, the structure may be a circle or a square. Alternatively, the structure may be of any arbitrary shape and size.

In one embodiment, the structures are formed from a thermoplastic polymer. The thermoplastic polymer may comprise monomers selected from the group consisting of acrylates, phthalamides, acrylonitriles, cellulosics, styrenes, alkyls, alkyls methacrylates, alkenes, halogenated alkenes, amides, imides, aryletherketones, butadienes, ketones, esters, acetals, carbonates and co-monomers thereof. In one embodiment, the thermoplastic polymer is selected from polycarbonate or poly methyl methacrylate.

Exemplary monomers to form the thermoplastic polymer may be selected from the group consisting of methyls, ethylenes, propylenes, methyl methacrylates, methylpentenes, vinylidene, vinylidene chloride, etherimides, ethylenechlorinates, urethanes, ethylene vinyl alcohols, fluoroplastics, carbonates, acrylonitrile-butadiene-styrenes, etheretherketones, ionomers, butylenes, phenylene oxides, sulphones, ethersulphones, phenylene sulphides, elastomers, ethylene terephthalate, naphthalene terephthalate, ethylene naphthalene and combinations thereof.

The substrate may be comprised of an organic material, such as a polymer, or an inorganic material, such as metal. The substrate may be planar or non-planar. Exemplary substrate materials include silicon, glass, quartz, mica, ceramics, polymers such as polyethylene, polypropylene, polyesters such as polyethylene terephthalate, polybutylene terephthalate, polyamides, fluropolymers and polysulphone and metals such as gold, silver, copper. Preferably, the substrate is formed from silicon.

There is also provided an array comprising a substrate having an array of structures projecting therefrom, wherein each of said structures have a defined topography thereon, and wherein at least one structure has a different topography from at least one other structure.

Advantageously, the array may be replicated using known casting methods, for example PDMS casting to form PDMS or hard PDMS replicas of the array made by the method as described herein. This provides a cost-effective way of producing replicas of the original array.

There is also provided the use of an array according to the invention as a microarray for studying cells. Advantageously, the microarray may be used in high throughput screening for cell response studies (cell adhesion, cell differentiation, cell proliferation, and gene expression), protein uptake experiments and bacterial adhesion studies and the like.

There is also provided an array according to the invention wherein said array is made in accordance with the method as described herein.

There is also provided a microarray having a substrate body comprising a substrate having an array of structures projecting therefrom, wherein each of said structures have a defined topography thereon, and wherein at least one structure has a different topography from at least one other structure.

In one embodiment, for easy reference to the locations of different topographies on the array, a document “map” that identifies the location of said structures comprising different topographical formations on said array may be provided. In one embodiment, as a reference point for easy identification of the location of the different topographical formations, the first column of the said structures on the array may be displaced further from an edge of the array than the last column of structures. This may then be correlated with the document map in order to locate particular topographical formations disposed on the array.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1A-FIG. 1C are schematic diagrams showing a process for producing an array as described herein.

FIG. 2 is a photograph showing a working prototype of the array made according to an embodiment of the present disclosure.

FIG. 3A-FIG. 3J show Scanning Electron Microscopy (SEM) images of the different topographies of an array of the present disclosure.

FIG. 4A-FIG. 4C shows representative Atomic Force Microscopy (AFM) section analyses of different designs on the array, demonstrating some of the different co-existing height topographies achievable on the array.

FIG. 5 is a photograph showing a Poly(dimethylsiloxane) (PDMS) replica of the design from an array according to the present disclosure.

FIG. 6 is a graph showing the percentage of Tull positive vs GFAP positive cells attached on various topographies of an array of the present disclosure.

FIG. 7 is a graph showing the percentage of BrdU incorporation for Human Mesenchymal Stem cells attached on various topographies of an array of the present disclosure.

FIG. 8A and FIG. 8B are graphs showing the amount of IL-2 secretion for T cells attached on various topographies of an array of the present disclosure.

FIG. 9A and FIG. 9B are graphs showing the percentage of CD44⁺Cd24^{-low}ESA⁺ cancer cells attached on various topographies.

### DETAILED DESCRIPTION OF DRAWINGS

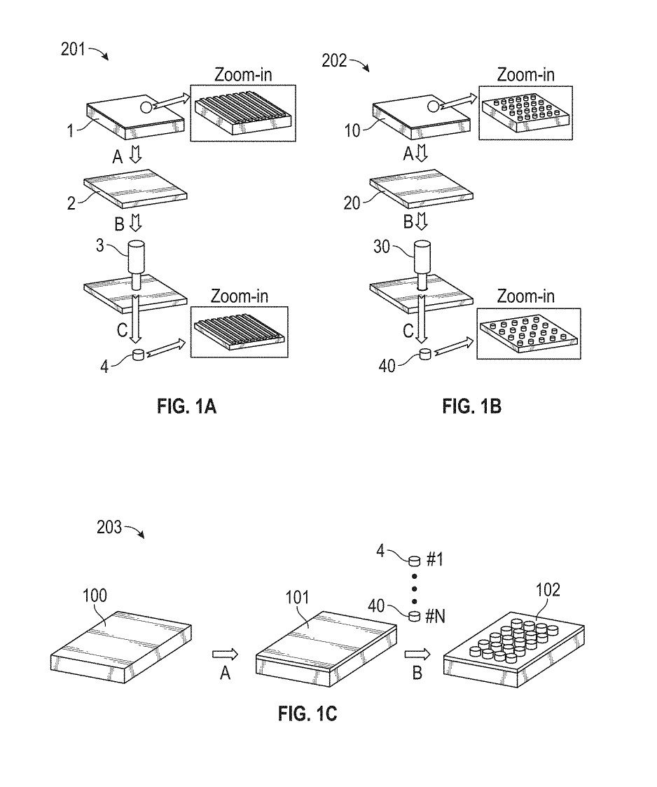

FIG. 1A shows a schematic diagram of a method 201 for making a topographical structure in the form of a grating pattern onto a sacrificial substrate polymer film (steps (A) to (C)).

In Step (A) of FIG. 1A, there is shown a Nano-Imprint Lithography (NIL) step to form a substrate having a topographical formation in the form of imprints as shown in the inset. NIL is a known technique to persons skilled in the art. In the NIL technique, the mold 1 is pressed into the surface of the substrate in the form of substrate 2, at a temperature of 180° C. and pressure of 60 Bars for 600 s to form a topographical structure on the substrate 2.

In Step (B) of FIG. 1A, the substrate 2 is cooled before demolding the substrate 2 from the mold 1.

In Step (C) of FIG. 1A, a portion of the topographical formation is removed from the substrate 2 using a tissue punch 3 to form a structure 4 having a topographical formation thereon for subsequent use in making an array.

FIG. 1B shows a schematic diagram of a method 202 for fabricating a topographical formation in the form of a pillar pattern onto a sacrificial substrate polymer film (steps (A) to (C)).

In Step (A) there is shown a Nano-Imprint Lithography (NIL) step to form a substrate having a topographical formation in the form of an ordered array as shown in the inset. In the NIL method step A of FIG. 1B, a preheating step is first performed at 180° C. for 120 s. Then the mold 10 is pressed into the surface of the sacrificial substrate in the form of substrate 20, at a temperature of 180° C. and a pressure of 40 Bars for 300 s to form a topographical formation on the substrate 20.

In Step (B) of FIG. 1B, the substrate 20 is cooled before demolding the substrate 20 from the mold 10.

In Step (C) of FIG. 1B, a portion of the topographical formation is removed from the substrate 20 using a tissue punch 30 to form a structure 40 for subsequent use in making an array.

FIG. 1C shows a schematic diagram of a method 203 for making an array 103 on a substrate 100, a process whereby the structures produced by methods 201 and 202 are adhered to the substrate 100.

In step (A) of FIG. 1C, PDMS mixed in a ratio of 3:1 (elastomer:curing agent) is first degassed using a vacuum for 15 min to remove any bubbles in the mixture. The PDMS is then spin-coated onto the silicon substrate 100 at 3000 rpm for 30 seconds. The resultant layer of PDMS 101 acts as a bonding material to adhere the structures 4, 40 onto the substrate 100.

In step (B) of FIG. 1C, the structures 4, 40 having topographical formations (1# to N#) produced by method 201 and 202 are adhered to the PDMS layer 101 in a matrix arrangement. The PDMS is further degassed under vacuum conditions to remove any residual bubbles remaining in the PDMS layer 101. The PDMS layer 101 is then cured by heating the assembled substrate in a vacuum oven at 70° C. for 1 h. This adheres the structures 4, 40 to the substrate firmly 100, resulting in an array 102 according to the present disclosure.

### EXAMPLES

Non-limiting examples of the invention and a comparative example will be further described in greater detail by reference to specific Examples, which should not be construed as in any way limiting the scope of the invention.

### Example 1

This example illustrates a process of the present disclosure for fabricating an array in accordance with the present disclosure using methods 201-203 as depicted in FIG. 1A-FIG. 1C.

To demonstrate the wide range of different topographies which could be incorporated on a single substrate, a mixture of designs was used. The various topographies include, but are not limited to: gratings, pillars and dimples, curved lens structures as well as complex three-dimensional hierarchical structures, with different resolutions and aspect ratios.

FIG. 1A and FIG. 1B respectively illustrate a process for producing a topographical formation with a grating pattern and a pillar pattern.

Thermal nanoimprint lithography was first used to fabricate various topographical formations separately onto individual substrates 2, 20 (for example, Polycarbonate (PC) or Poly (methyl methacrylate film) (PMMA). Referring to FIG. 1A for example, molds 1, 10 bearing the desired topography were pressed into the surface of the substrates 2, 20 at various optimized temperatures, pressures and durations depending on the desired topography, in order to imprint the desired pattern onto the sacrificial substrates 2, 20. For gratings, dimples and curved lens structures, a single imprinting condition was used: 180° C., 60 Bars, 600 s. For delicate structures such as pillar structures, a preheating step was performed at 180° C. for 120 s before the imprinting at 180° C., 40 Bars, 300 s to allow high yield imprinting of such structures. For hierarchical structures, a sequential imprinting process was employed: 180° C., 60 Bars, 600 s for the primary imprinting process; followed by 140° C., 40 Bars, 300 s for the secondary imprinting process.

Next, the substrates 2, 20 were cooled before they were demolded from their respective molds 1, 10. A Tissue puncher 3, 30 was then used to cut out a uniform 2 mm diameter sized portion of the substrate 2, 20 resulting in the production of structures 4, 40 bearing the desired topographical formations.

The above process was repeated for different topographical formations, thereby producing “N” number of topographical formations (indicated as #1-#N, in FIG. 1C).

FIG. 1C illustrates the process of making an array in accordance with the disclosure. Firstly, PDMS (Sylgard®184 mixed in a ratio of 3:1 (elastomer:curing agent)) was degassed in a vacuum for 15 minutes to remove any bubbles in the mixture. The PDMS was then spin-coated onto a silicon substrate 100 at 3000 rpm for 30 s. The resultant layer of PDMS 101 acts as a bonding material for securing the structures to the substrate 100.

Next, the structures with various topographical formations were assembled manually using tweezers onto the PDMS layer 101 in a matrix arrangement The PDMS layer acts as a bonding layer to adhere the structures onto the substrate. Further degassing was then performed using under vacuum conditions to remove residual bubbles remaining beneath the PDMS layer 101. The PDMS layer 101 was then cured by heating the assembled substrate in a vacuum oven at 70° C. for 1 h. This bonded the topographical structures (#1-#N) firmly onto the substrate 100, thereby producing an array 102 of the current invention.

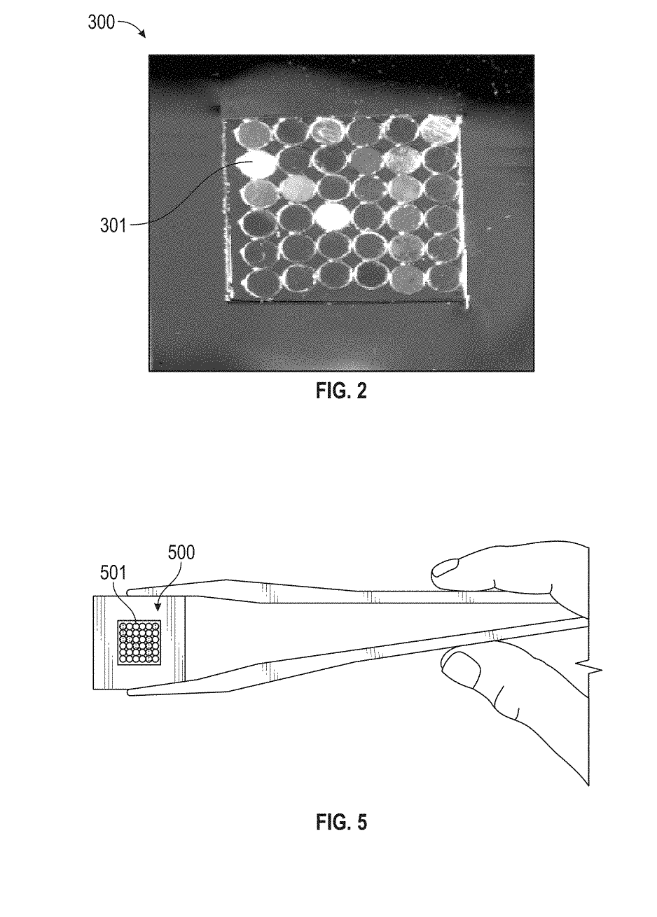

FIG. 2 shows a working prototype 300 of an array fabricated using the above process. The arrow points to an example of a 2 mm topographical structure 301. The prototype 300 consists of a matrix of 6×6 structures 301 having different topographical formations thereon.

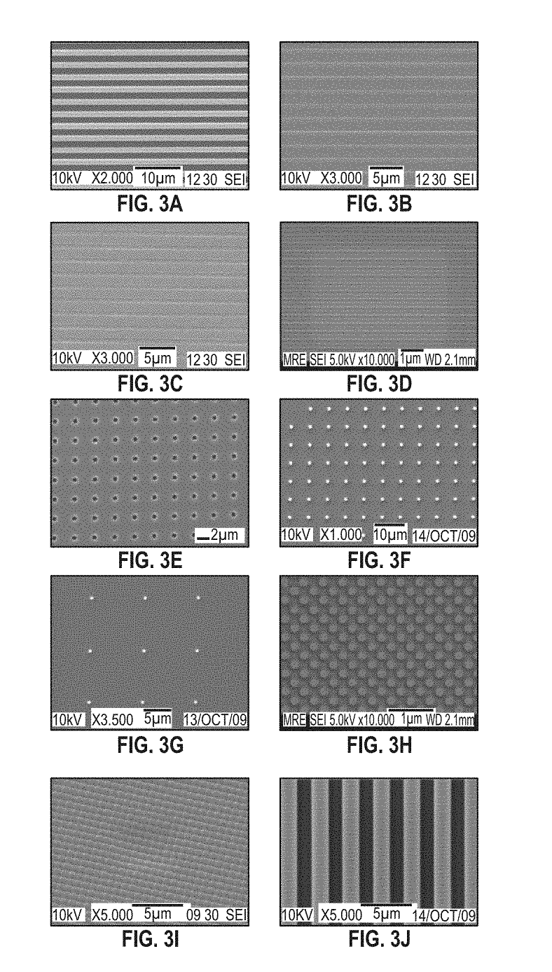

SEM images of some of the different topographical formations that were fabricated are shown in FIG. 3A-FIG. 3J. The topographies shown are: FIG. 3A 2 μm line, 2 μm space; FIG. 3B 1 μm line, 2 μm space; FIG. 3C 2 μm line, 1 μm space; FIG. 3D 250 nm line 250 nm space; FIG. 3E 1 μm dimple, 9 μm pitch; FIG. 3F 2 μm pillars, 12 μm pitch; FIG. 3G 500 nm pillars, 10 μm pitch; FIG. 3H 250 nm pillars, 500 nm pitch; FIG. 3I 1 μm pitch microlens; FIG. 3J 2 μm line 2 μm space ⊥ 250 nm line 250 nm space.

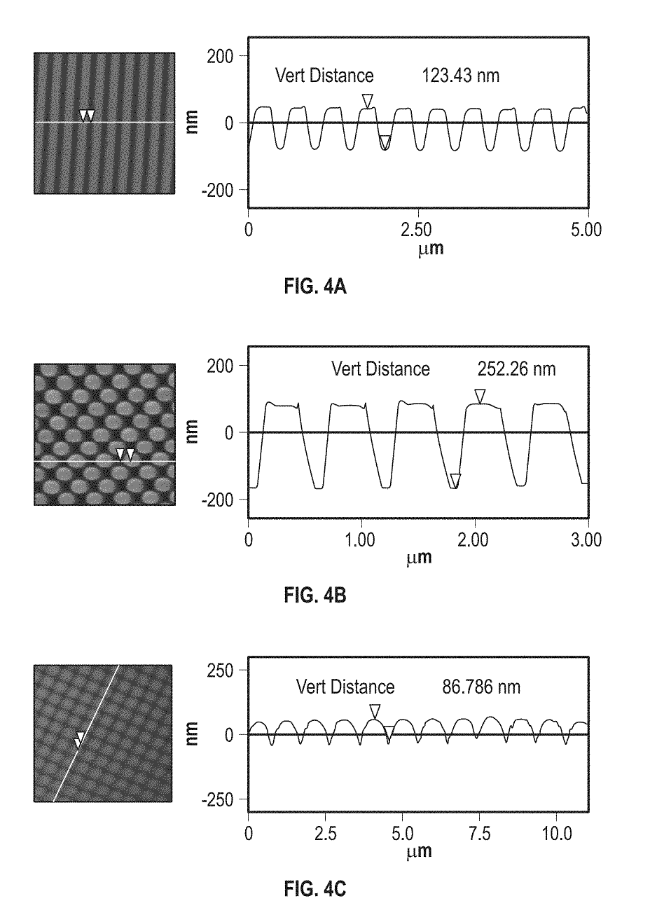

FIG. 4A-FIG. 4C shows a range of topographical formations with different heights, clearly demonstrating the possibility of incorporating topographical formations of different aspect ratios onto a single array. The different topographies shown are: FIG. 4A 250 nm line 250 nm space gratings with 120 nm height; FIG. 4B 250 nm pillars, 500 nm pitch with 250 nm height; FIG. 4C 1 μm pitch microlens with 90 nm sag.

The array of the present disclosure can also be used to form replica copies of the design by using a conventional PDMS casting method for example. A PDMS replica copy 500 of the design from an array is shown in FIG. 5. The replica array 500 has an array of structures 501 having different topographical formations thereon.

### Example 2

In this example, an array as described herein was used to illustrate an application to facilitate fast microscopy based screening to investigate neural precursor cell differentiation enhancement with respect to various topographies.

Neural progenitor cells were isolated from the hippocampal region of a postnatal day 5 mouse brain. They were expanded on the tissue culture plate and coated with 8 μg/ml of natural mouse laminin (Invitrogen Pte. Ltd.) in a proliferation medium composed of DMEM/F12 (Biological Industries), N2 supplements (GIBCO, Invitrogen Pte Ltd) and Penicillin/Streptomycin. Growth factors, basic fibroblast growth factor, bFGF, (GIBCO, Invitrogen Pte. Ltd.), and epidermal growth factor, EGF, (Invitrogen Pte. Ltd.), were added freshly at a final concentration of 20 ng/ml to the expansion medium.

Polydimethyl siloxane (PDMS) substrates were replicated from an array master mold using a 10:1 ratio of elastomer and curing agent (SYLGARD®184 Elastomer Kit, Dow Corning). The elastomer and the curing agent were mixed thoroughly at a ratio of 10:1 and the mixture was degassed before pouring onto the microarray chip. Degassing was repeated subsequently for 45 minutes until there were no residual visible bubbles. The curing was completed by keeping the sample inside a curing oven at a temperature of 65° C. overnight. The replica array was peeled off from the PDMS substrate.

For neural differentiation, the PDMS replica substrates were plasma treated and coated consecutively with Poly-L-ornithine (Sigma Aldrich) and laminin at 20 μg/ml overnight. The cells (Passage 12-17) were detached from the tissue culture plate and seeded on the PDMS substrates at a density of 7,500 cells/cm². The differentiation medium during the first phase consisted of DMEM/F12, N2 supplements, Penicillin/Streptomycin and B27 supplements (GIBCO, Invitrogen Pte. Ltd.) with bFGF at a concentration of 5 ng/ml. After 7 days, the differentiation medium was changed to the second phase medium made up of neurobasal medium (Invitrogen Pte. Ltd.) supplemented with B27 and 0.25× N2 and mixed at a ratio of 1:1 with normal Neural Precursor Cell (NPC) expansion medium without N2 supplements and bFGF.

At the end of the time point, the cells were fixed in 4% paraformaldehyde and immunocytochemical staining was performed with the following primary antibodies with their respective optimized concentrations: anti-rabbit β-tubulin III or Tuj1 (Tuj1=neuron-specific class III beta tubulin) antibody (1:600, Sigma Aldrich); anti-mouse glial fibrillary acidic protein, GFAP, antibody (1:600, Chemicon, Millipore Pte Ltd). The secondary antibodies were purchased from Invitrogen Pte Ltd: Alexor Fluor 546 Goat anti-mouse IgG (H+L) and Alexor Fluor 488 Goat anti-rabbit IgG (H+L) both at the concentration of 1:750. Nuclei were counterstained with 4′,6-diamidino-2-phenylindole, DAPI, (Invitrogen).

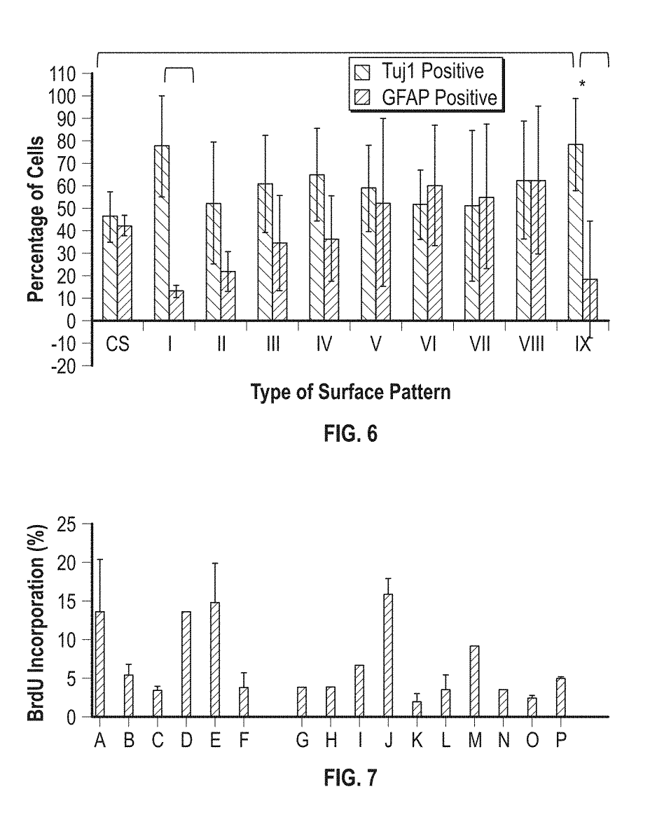

Imaging was performed with a Leica fluorescent microscope equipped with Q imaging camera (Canada) and with the software, Q capture Pro. From each topographical formation, 7-10 images were captured and image analysis was done using the software, Image J (NIH). Statistical analysis with one way ANOVA was done using Microsoft Excel after compiling the raw data. The percentage of neural precursor cells which differentiated into immature neurons (positive for Tuj1 staining) and astrocytes (positive for GFAP staining) was compared with respect to different topographical formations as well as coverslip control samples (CS). FIG. 6 shows the proportion of immature neurons vs astrocytes attached to different topographical formations. The different topographies compared were: CS: control; I: 2 μm line, 2 μm space; II: 1 μm line, 2 μm space; III: 2 μm line, 1 μm space; IV: 250 nm space, 250 nm space; V: 1 μm diameter holes, 9 μm pitch; VI: 2 μm diameter pillars, 12 μm pitch; VII: 500 nm diameter pillars, 10 μm pitch; VIII: 130 nm diameter pillars, 400 nm pitch; IX: 2 μm line, 2 μm space ⊥ 250 nm line, 250 nm space (hierarchy). The error bars in FIG. 6 represent standard deviations from the mean.

As can be seen from FIG. 6, a significant difference in the proportion of immature neurons vs astrocytes was observed for topographies I and IX.

### Example 3

In this example, an array as described herein was used to illustrate an application to facilitate fast microscopy based screening to investigate the regulation of Human Mesenchymal Stem Cell proliferation with respect to various topographical formations.

BrdU incorporation was used as an indicator of DNA synthesis and therefore a proxy for measuring the extent of cell proliferation.

Polydimethyl siloxane (PDMS) substrates were replicated from an array in the form of a master mold using a 10:1 ratio of elastomer and curing agent (SYLGARD®184 Elastomer Kit, Dow Corning). The elastomer and the curing agent were mixed thoroughly at a ratio of 10:1 and the mixture was degassed before pouring onto the array chip. Degassing was repeated subsequently for 45 minutes until there was no residual visible bubble. The curing was completed by keeping the sample inside a curing oven at a temperature of 65° C. overnight. The array was peeled off from the PDMS substrate. The PDMS replica array was then used in the cell assay. Human mesenchymal stem cells were cultured for 7 days on a PDMS stitch array replica and serum was withdrawn from serum-depleted cells for 6 hours on day 5.

On day 7, BrdU was added into culture medium (1:1000) and the samples were incubated for 4 hours. The cells were then fixed with 4% paraformaldehyde, permeabilized with 0.1% Triton-X100, further incubated in 4N HCl for 10 minutes and then blocked with 10% goat serum and 1% BSA in PBS. BrdU was labeled with mouse anti-human BrdU diluted 1:50 as primary antibody, Alexa-Fluor 546 conjugated goat anti-mouse antibody diluted 1:1000 as secondary antibody, and the nuclei were counterstained with 4′,6-Diamidino-2-phenylindole. Samples were then imaged with fluorescence microscope. Acquired fluorescence images were analyzed by Image J software. BrdU incorporation percentage was calculated as the percentage of BrdU positive-stained cells to the total number of cells on each of the different topographical formations of the replicated PDMS substrate.

FIG. 7 shows the results obtained. The topographies compared were: A: 2 μm line, 2 μm space; B: 2 μm line, 1 μm space; C: 1 μm line, 2 μm space; D: 250 nm line, 250 nm space, 125 nm height; E: 1 μm diameter pillars, 9 μm pitch; F: 2 μm diameter holes, 12 μm pitch; G: 500 nm diameter holes, 10 μm pitch; H: 130 nm diameter holes, 400 nm pitch; I: 2 μm line, 2 μm space ⊥ 250 nm line, 250 nm space (hierarchy); J: 2 μm line, 2 μm space//250 nm line 250 nm space (hierarchy); K: 2 μm line and 2 μm space+250 nm diameter holes (hierarchy); L: 250 nm diameter holes, 500 nm pitch; M: 250 nm line, 250 nm space, 250 nm height; N: 1 μm diameter concave lens, 1 μm pitch; O: 1 μm diameter convex lens, 1 μm pitch; P: 1.8 μm diameter concave lens, 2 μm pitch; Q: 1.8 μm diameter convex lens, 2 μm pitch. The percentage of BrdU incorporation indicates the extent of proliferation. In particular, the higher the percentage of BrdU incorporation, the greater the extent of Human Mesenchymal Stem Cell proliferation.

From the results it can be seen that narrower line widths of the topographical formation result in decreased stem cell proliferation. Secondly, topographies comprising pillars appear to significantly enhance proliferation. Lastly, the preliminary studies suggested that the line width may exert a more significant effect on the proliferation of Human Mesenchymal Stem Cells compared to the spacing between the lines.

### Example 4

In this example, an array as described herein was used to illustrate an application to facilitate fast microscopy based screening to investigate T-cell activation with respect to various topographical formations.

An array in accordance with the disclosure was used as a master mold to form replica copies of the array using a material termed hard PDMS: 3.4 g of 7-8% vinylmethylsiloxane, 1 g of 25-30% methylhydrosiloxane, 18 microliters of Platinum(0)-2,4,6,8-tetramethyl-2,4,6,8-tetravinylcyclotetrasiloxane), and 10 microliters of 2,4,6,8-Tetramethyl-2,4,6,8-tetravinylcyclotetrasiloxane were mixed and spin coated onto the array. Following hardening, a thick layer of PDMS (Sylgard®184, Dow Corning) with a mixture of (elastomer:curing agent of 10:1) was poured in order to allow easy handling of the replica.

The replica arrays were coated with antibodies to TCR and CD28 (receptors on the T cell surface that upon this binding provide activating and co-stimulatory signals, respectively). Naïve CD4+ T cells were isolated from mice and seeded onto these surfaces in RPMI media supplemented with 10% fetal bovine serum. T-cell activation was measured on the basis of secretion of IL-2, a cytokine indicative of T cell activation, over the course of six hours. IL-2 secretion was assayed using a commercial, surface-capture method from Miltenyi Biotech (IL-2 secretion detection kit). Fluorescence associated with IL-2 secretion from this assay was measured on a cell-by-cell basis by microscopy, and compared using Kruskal-Wallis non-parametric approaches.

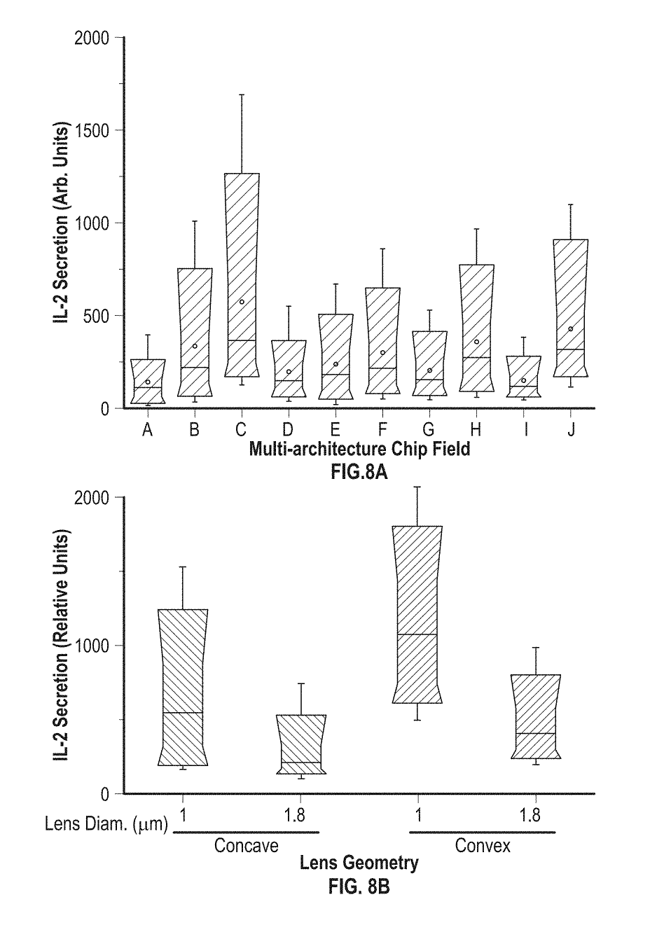

FIG. 8A and FIG. 8B illustrate the results obtained from the microscopy screening of a small subset of the topographical formations on the replica array. A-J in FIG. 8A are topographies—A: 2 μm line, 2 μm space; B: 2 μm line, 1 μm space; C: 1 μm line, 2 μm space; D: 250 nm line, 250 nm space, 250 nm height; E: 250 nm line, 250 nm space, 150 nm height; F: 460 nm line, 70 nm space, G: 2 μm diameter holes, 12 μm pitch; H: 500 nm diameter holes, 10 μm pitch; I: 250 nm diameter holes, 500 nm pitch; J: 130 nm diameter holes, 400 nm pitch.

FIG. 8B illustrates the results obtained using topographies of lens geometry consisting of: 1 μm diameter, 1 μm pitch concave lens; 1.8 μm diameter, 2 μm pitch concave lens; 1 μm diameter, 1 μm pitch convex lens; 1.8 μm diameter, 2 μm pitch convex lens.

Comparison of IL-2 secretion across the replica array revealed that T cells are able to recognize and respond differently to different topographies. Most prominently, these T cells showed higher levels of IL-2 secretion on surfaces presenting arrays of 1 μm diameter, 1 μm pitch lenses than those with 1.8 μm diameter, 2 μm pitch lens features. In addition, IL-2 secretion was higher on convex lenses rather than concave ones (such as those illustrated in FIG. 8B).

A further potential extension of this application can be achieved by using this array for immunotherapy. T-cells can be extracted from patients, activated and expanded in cell culture for sufficient number of cells for therapeutic purposes with an array of specific topography. The activated T-cells are injected back to the patient for immunotherapy. As the substrate will not be transferred to the patients, there will be lower side-effects and toxicity. Thus this protocol/system will have limited regulatory barriers and can be brought to clinical trials very rapidly.

### Example 5

This example was used to illustrate a potential application of using the microarray, as a unique, fast and low cost marker for cancer diagnostic applications, since different subpopulations of cancer cells display different adhesion responses to specific topographical formations.

This example shows that different topographical formations can influence the adhesion response of different sub-populations of cancer cells.

This example was not carried out on a replica array chip. This example has been included to illustrate that 1) different topographies can influence the response of cancer cells, and 2) since the microarray consists of a variety of different topographical formations, it can be potentially used as a cancer diagnostic tool.

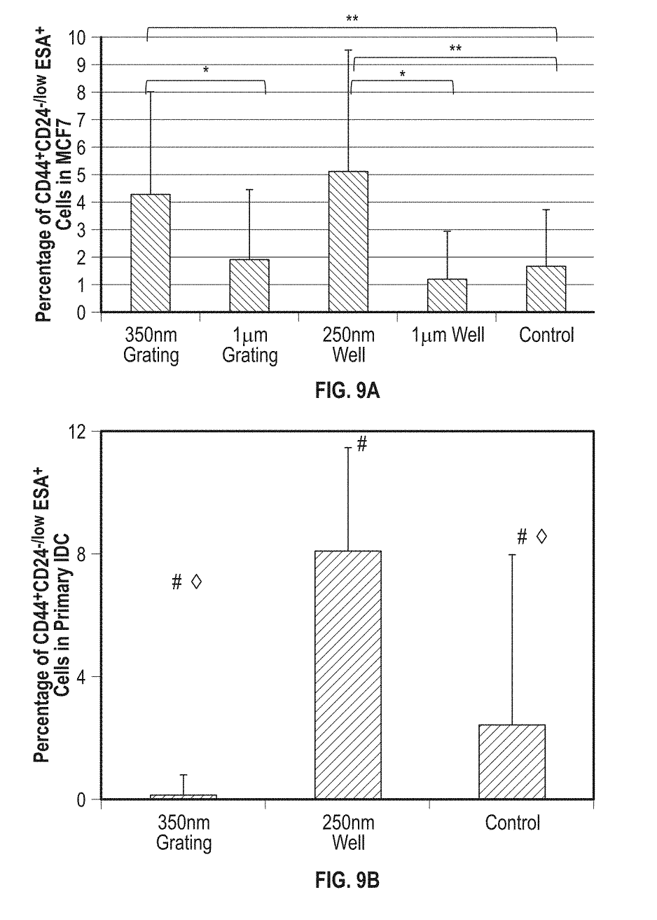

FIG. 9A shows the results obtained for the breast cancer cell line MCF7 and FIG. 9B shows the results for human primary invasive ductal carcinoma (IDC) cells.

The results were analysed by ANOVA, which showed that there was a significant difference for all the topographies as compared to the control (flat surface/no pattern surface). For MCF7 cells, t-tests showed a preferential isolation of CD44⁺CD24^{-/low}ESA⁺MCF7 cells on the nano-scale features (*p<0.05, mean±SD, n=3) as compared to the micro-scale features (see FIG. 9A).

In the IDC cell study, significantly more primary cells adhered onto the 250 nm well (#p<0.001, mean±SD, n=3) as compared to the control (see FIG. 9B).

### Culturing of MCF7 Cells

The human breast cancer cell line, MCF7, was obtained from the American Type Culture Collection (ATCC) and maintained using Dulbecco's modified Eagle's medium (DMEM, Sigma Aldrich) supplemented with 10% fetal bovine serum (FBS), 0.01 mg/ml bovine insulin, 1% penicillin streptomycin and 1% non-essential amino acids solution. The medium was changed every three days.

### Culturing of Invasive Ductal Carcinoma (IDC) Cells

IDC cells were obtained from the NUH-NUS Tissue Repository. These cells were derived from breast tumor tissue donated by patients under the approval of NUS Institutional Review Board. The cells were maintained in culture using mammary epithelial growth medium (MEGM, Lonza) supplemented with 10% FBS, amphotericin B and gentamicin.

### Cell Seeding on Micro- or Nano-Topographical Substrates

Poly-L-lactic substrates with nanotopography patterns were cut to 1.0 cm by 1.0 cm dimensions. They were submerged in 70% ethanol solution and exposed to ultraviolet light simultaneously for 20 minutes for sterilization.

Either MCF7 or IDC cells were seeded onto the PLLA films with a seeding density of about 10,000 cells/cm².

After 4 and 24 hours of culturing MCF7 cells on the PLLA films, the cells were fixed with 4% paraformaldehyde and immuno-fluorescence stain for the stem cell markers with standard protocol, using primary antibodies specific for human cell surface markers CD44, CD24 and epithelial cell adhesion molecule (EpCAM or ESA). The primary antibodies used were: rat anti-CD44 FITC (Abcam), mouse anti-CD24 PE (Abcam) and rabbit anti-ESA (Santa Cruz). The secondary antibodies used were: goat anti-mouse Alexa Fluor 546, goat anti-rabbit Alexa Fluor 647 and rabbit anti-rat FITC. The samples were then mounted on glass slides with fluoromount (Sigma) and imaged with a fluorescence microscope at 20×, 40× and 63× magnification. Flow cytometry was also performed using the antibodies for CD44, CD24 and ESA. The breast cancer stem cells were identified as the CD44⁺/CD24⁻/ESA⁺ population.

### APPLICATIONS

The disclosed process may be applied in numerous industrial applications, not least in therapeutic applications, pharmaceutical compositions. The fabrication of different topographies on a single substrate surface may be used for fast and efficient experimental studies investigating the effect of different surface topographies on a wide range of cell types. The array and its PDMS replica can also be used for fast microscopy screening for cell-topography interactions, for immunotherapy and cancer diagnostic applications and immunotherapy applications.

Advantageously, the disclosed process may be highly reproducible and may be used to simultaneously fabricate a large range of different topographies, such as different heights, dimensions and feature shapes onto a single substrate surface.

Advantageously, the method described herein provides a low cost fabrication process. In particular, nanoimprint technology, which is a cost-efficient, scalable, high throughput nanoimprinting technique, is used to create the different topographies which are otherwise prohibitively expensive if they are fabricated by conventional electron beam lithography.

It is a further advantage that the method as described herein provides a biocompatible array.

It is a further advantage of the method as described herein that the array may be replicated using known casting methods, for example PDMS casting. This provides a cost-effective way of producing replicas of the original array.

Advantageously, the method as described herein does not encounter problems of resolution limit in providing the different topographies, as nanoimprinting is used to form the topographies, where the limit of resolution is limited by the mold to be used for imprinting.

It is a further advantage of the method as described herein that the individual topographies of the array can be optimized as different topographies are imprinted separately.

It is a further advantage of the method as described herein that a dry/etch-free approach is used to form the different topographies on a single substrate.

Advantageously, the method as described herein permits the use of different types of polymer (for example, PMMA, PC, ETFE and the like) to form the structures having a pre-defined topography thereon.

Advantageously, the array of the invention can also be used to form replica copies of the array design by using various methods well known in the art, such as a conventional PDMS casting method.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-57. (canceled)

58. A method of making an array for cell assays comprising the step of providing an array of structures that each extend from a substrate and having a pre-defined topography thereon, wherein at least one structure (A) has a different topography from at least one other structure (B), wherein structure (A) is imprinted separately to structure (B), and wherein structure (A) is capable of being imprinted at an imprinting condition (A_{C}) that is different to imprinting condition (B_{C}) of structure (B).

59. The method as claimed in claim 58, wherein the structures are generally elongate and have a proximal end that extends from the substrate and a distal end that is opposite to said proximal end.

60. The method as claimed in claim 59, wherein the topography of said structures is provided on said proximal end.

61. The method as claimed in claim 60, wherein the elongate structures have a longitudinal axis extending from their proximal end to their distal end and wherein said longitudinal axis is generally normal to a planar surface of said substrate.

62. The method as claimed in claim 58, wherein the providing step comprises the step of adhering said structures to said substrate.

63. The method as claimed in claim 62, wherein said adhering step comprises the step of providing an adhesive layer on said substrate before said adhering step.

64. The method as claimed in claim 63, wherein said providing an adhesive layer on said substrate comprises the step of spin-coating a polymer solution onto said substrate.

65. The method as claimed in claim 64, removing the polymer solution while said array of structures are disposed in said polymer solution to form a polymer layer that adheres said structures to said substrate.

66. The method as claimed in claim 58, wherein said topography is formed by formations in the microscale or the nanoscale size range.

67. The method as claimed in claim 66, comprising, before said providing step, the step of forming said topographic formations on a sacrificial substrate.

68. The method as claimed in claim 67, wherein the step of forming said topographic formations on a substrate comprises an imprint lithography step.

69. The method as claimed in claim 68, wherein the step of forming said topographic formations comprises the step of providing the sacrificial substrate with an array of the topographic structures in the microscale or nanoscale size range.

70. The method as claimed in claim 69, wherein the array of microscale or nanoscale topographic structures on said sacrificial substrate are selected from the group consisting of trenches, pillars, gratings, dimples, wells, and other 3-dimensional structures.

71. The method as claimed in claim 69, comprising the step of removing a portion of said microscale or nanoscale topographic structures while attached to said sacrificial substrate.

72. The method as claimed in claim 58, wherein said providing step comprises providing an ordered array of structures on said substrate.

73. The method as claimed in claim 58, wherein said structures are formed from a thermoplastic polymer.

74. The method as claimed in claim 73, wherein said thermoplastic polymer comprises monomers selected from the group consisting of acrylates, phthalamides, acrylonitriles, cellulosics, styrenes, alkyls, alkyls methacrylates, alkenes, halogenated alkenes, amides, imides, aryletherketones, butadienes, ketones, esters, acetals, carbonates and co-monomers thereof.

75. The method as claimed in claim 70, wherein the gratings and dimples are imprinted under an imprinting condition of a temperature of 180° C. at 60 bars for 600 seconds.

76. The method as claimed in claim 70, wherein the pillars are preheated, prior to imprinting, at 180° C. for 120 seconds and imprinted under an imprinting condition of a temperature of 180° C. at 40 bars for 300 seconds.

77. The method as claimed in claim 70, wherein the topographic structures are hierarchically imprinted under a first imprinting condition of a temperature of 180° C. at 60 bars for 600 seconds and a second imprinting condition of a temperature of 140° C. at 40 bars for 300 seconds, wherein the first imprinting condition and the second imprinting condition are performed sequentially.
